(19) United States
(12) Patent Application Publication
(10) Pub. No.: US 2021/0127765 A1
(43) Pub. Date: May 6, 2021

(54) ACCESS TO DIALYSIS SITE FOR A FULL-ZIP HOODIE

(71) Applicant: Nathan Thompson, JR., Lansing, MI (US)

(72) Inventor: Nathan Thompson, JR., Lansing, MI (US)

(21) Appl. No.: 17/085,490

(22) Filed: Oct. 30, 2020

Related U.S. Application Data
(60) Provisional application No. 62/928,240, filed on Oct. 30, 2019.

(51) Int. Cl. A41D 13/12

(52) U.S. Cl. CPC A41D 13/1245; A61M 1/14

(57) ABSTRACT

A garment for use by a dialysis patient is presented. The garment includes a right sleeve, a left sleeve and a plurality of zipper systems. The zipper systems are configured to provide access to a predetermined site of the arm of a dialysis patient. The garment is structured and arranged as a hooded top sweatshirt. The garment is useful to provide comfort and warmth for a dialysis before, during, and after a dialysis treatment session.

### CROSS-REFERENCE TO RELATED APPLICATIONS

This application claims the benefit of U.S. Provisional Patent Application No. 62/928,240, filed on Oct. 30, 2019, which is incorporated by reference herein in its entirety.

### FIELD OF THE INVENTION

This invention relates to garments. More particularly, it relates to providing access to dialysis sites when wearing a garment.

### BACKGROUND

A hoodie (also spelled hoody) is a sweatshirt or a jacket with a hood. Hoodies often include a muff sewn onto the lower front, and (usually) a drawstring to adjust the hood opening.

Throughout the U.S., it is common for middle-school, high-school, and college students to wear sweatshirts—with or without hoods—that display their respective school names or mascots across the chest, either as part of a uniform or personal preference. Hoodies have become mainstream fashion in the U.S., transcending the clothing item's original utilitarian purpose, similar to jeans. This clothing item has found its way into a variety of styles, even so far as to be worn under a suit jacket.

In medicine, dialysis is the process of removing excess water, solutes, and toxins from the blood in people whose kidneys can no longer perform these functions naturally. This is referred to as renal replacement therapy.

Dialysis is used in patients with rapidly developing loss of kidney function, called acute kidney injury (previously called acute renal failure), or slowly worsening kidney function, called Stage 5 chronic kidney disease, (previously called chronic kidney failure and end-stage renal disease and end-stage kidney disease).

Dialysis is used as a temporary measure in either acute kidney injury or in those awaiting kidney transplant and as a permanent measure in those for whom a transplant is not indicated or not possible.

In research laboratories, dialysis technique can also be used to separate molecules based on their size. Additionally, it can be used to balance buffer between a sample and the solution “dialysis bath” or “dialysate” that the sample is in. For dialysis in a laboratory, a tubular semipermeable membrane made of cellulose acetate or nitrocellulose is used. Pore size is varied according to the size separation required with larger pore sizes allowing larger molecules to pass through the membrane. Solvents, ions and buffer can diffuse easily across the semipermeable membrane, but larger molecules are unable to pass through the pores. This can be used to purify proteins of interest from a complex mixture by removing smaller proteins and molecules.

When wearing a hoodie and attending a dialysis session, it is often difficult to access the desired site for dialysis. The hoodie has to be pulled over the head from the bottom of the hoodie. This can cause issues of time and energy as well as frustration by the person having to undergo dialysis.

In light of the foregoing, it would be desirable to devise an improved hoodie that is a full-zip hoodie to have easy access to a desired site for dialysis. Other advantages will become apparent in the disclosure.

### BRIEF DESCRIPTION OF THE DRAWINGS

FIG. 1 is an illustrated view of a garment for use by a dialysis patient.

FIG. 2 is an illustrated view of a right sleeve of the garment for use by a dialysis patient shown in FIG. 1.

FIG. 3 is an illustrated view of a left sleeve of the garment for use by a dialysis patient shown in FIG. 1.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

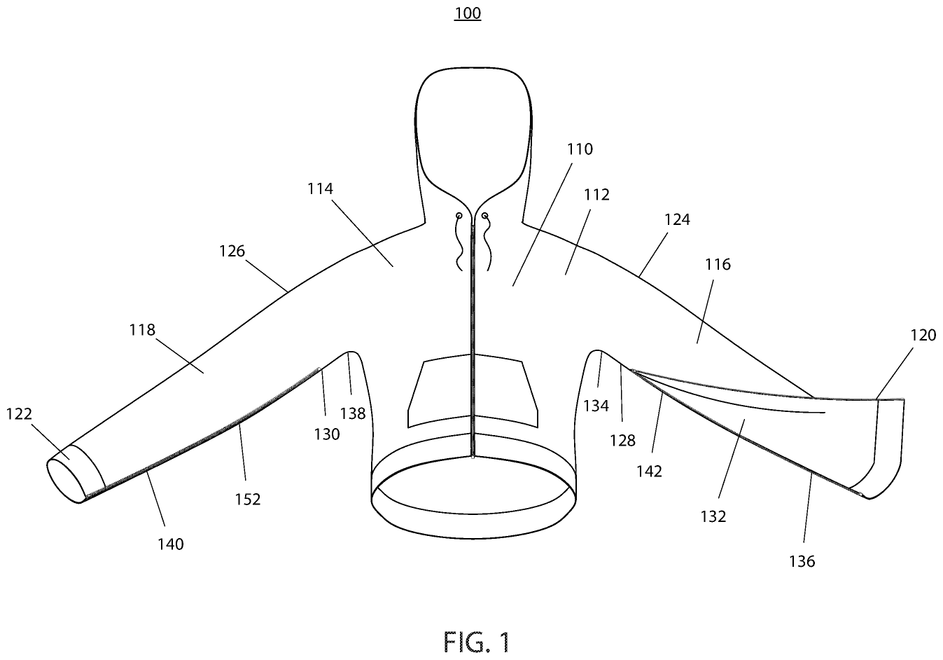

Referring to FIG. 1, a garment 100 for use by a dialysis patient is presented. The garment 100 may include a body 110, a right sleeve 118, a left sleeve 116, and a plurality of zippers including a left zipper system 142 and a right zipper system 152. The right sleeve 118 may be coupled to a right side 114 of the body 110 and the left sleeve 116 may be coupled to a left side 112 of the body 110. The right sleeve 118 may also include a right sleeve cuff 122, a right sleeve top surface 126, and a right sleeve underside 130 having a right sleeve underside upper portion 138 and a right sleeve underside lower portion 140. The left sleeve 116 may also include a left sleeve cuff 120, a left sleeve top surface 124, and a left sleeve underside 128 having a left sleeve underside upper portion 134 and a left sleeve underside lower portion 136. As illustrated, the left sleeve 116 also includes a left sleeve opening 132 that exposes the arm of a user for convenience during a dialysis treatment.

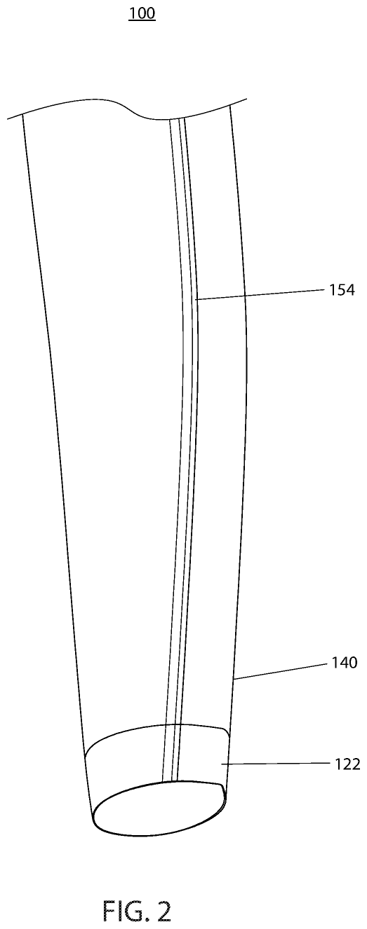

Referring now to FIG. 2, an illustrated view of the right sleeve 118 of the garment 100 for use by a dialysis patient shown in FIG. 1 is presented. The right sleeve 118 may include a double stitched hem 154 extending along the right sleeve underside lower portion 140 towards the right sleeve cuff. The right sleeve double stitched hem 154 may cover the right zipper system 152, thereby keeping the right zipper system 152 (not shown) discrete and protected while in a closed configuration.

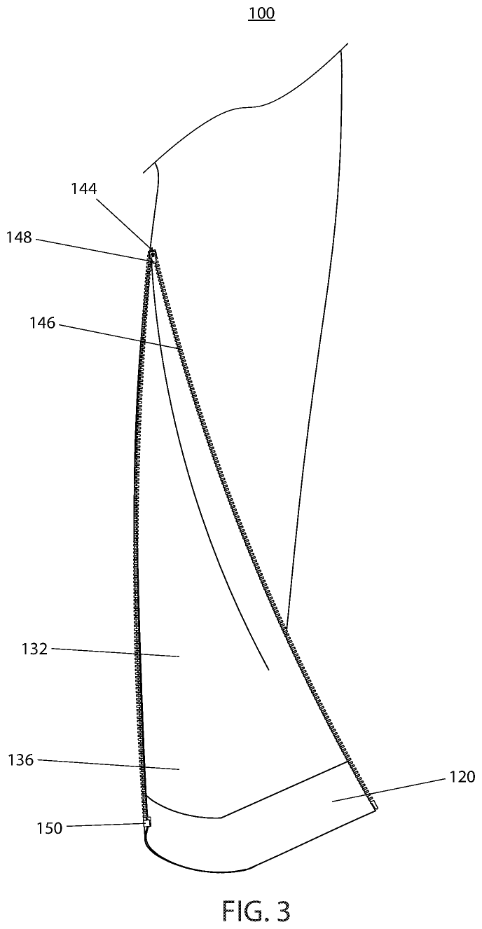

Referring finally to FIG. 3, an illustrated view of a left sleeve 116 of the garment 100 for use by a dialysis patient shown in FIG. 1 is presented. In this illustration, the left sleeve 116 is in an open configuration, thereby exposing the left sleeve opening 132. The left sleeve 116 of the garment 100 for use by a dialysis patient may include the left zipper system 142 having a left slider body 144, a left chain 146, a left pull tab 148, and a left retainer box 150. The left zipper system 142 may extend along the left sleeve underside lower portion 136 down towards the left sleeve cuff 120. When in an open configuration, the left sleeve opening 132 may provide access to a predetermined site on an arm of a patient when undergoing dialysis.

To use the garment for use by a dialysis patient, the user would simply put on the garment in a manner consistent with wearing a standard hoodie/sweatshirt-type garment. Next, the user may unzip one or both of the plurality of zipper systems, thereby exposing the respective left and right sleeve openings. The user may then pull back the opened sleeve(s) over the respective shoulders, thereby providing access to the desired dialysis site(s).

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A garment for use by a dialysis patient, said garment comprising:
a body;
a right sleeve, said right sleeve coupled to a right side of said body;
a left sleeve, said left sleeve coupled to a left side of said body;
a right sleeve cuff, said right sleeve cuff located at an end of said right sleeve;
a left sleeve cuff, said left sleeve cuff located at an end of said left sleeve;
a plurality of zipper systems, each of said zipper systems located on an underside of said right sleeve and on an underside of said left sleeve;
wherein in an open configuration said garment provides access to a predetermined site on an arm of a patient when undergoing dialysis; and
wherein in a closed configuration said garment covers access to said predetermined site.

2. The garment of claim 1, wherein the garment is a hooded top.

3. The garment of claim 2, wherein the hooded top is a zipped hooded top.

4. The garment of claim 1, wherein the garment is a hooded sweatshirt.

5. The garment of claim 4, wherein the hooded sweatshirt is a zipped hooded sweatshirt.

6. The garment of claim 1, wherein the garment is a hooded jacket.

7. The garment of claim 6, wherein the garment is a zipped hooded jacket.

8. The garment of claim 1, wherein the garment is a top.

9. The garment of claim 1, wherein the garment is a sweatshirt.

10. The garment of claim 1, wherein the garment is a jacket.

11. The garment of claim 1, wherein the garment further comprises a right sleeve opening.

12. The garment of claim 11, wherein said right sleeve opening is located at a right sleeve underside.

13. The garment of claim 12, wherein the garment has a left sleeve opening.

14. The garment of claim 13, wherein said left sleeve opening is located at a left sleeve underside.

15. The garment of claim 11, wherein said right sleeve opening extends from a right sleeve underside upper portion to said right sleeve cuff.

16. The garment of claim 13, wherein said left sleeve opening extends from a left sleeve underside upper portion to said left sleeve cuff.

17. The garment of claim 1, wherein each of said zipper systems includes a slider body, a chain, a pull tab, and a retainer box.

18. The garment of claim 1, wherein said garment is made of a 100% pure organic material.

19. The garment of claim 14, wherein when said garment is in said closed configuration a double stitched hem covers each of said zipper systems.

20. The garment of claim 1, further comprising a double stitched hem configured to hide visibility of each of said zipper systems when in said closed configuration.
